(19) United States
(12) Patent Application Publication
(10) Pub. No.: US 2006/0159622 A1
(43) Pub. Date: Jul. 20, 2006

(54) Pharmacodynamic assay for inhibitors of 11-beta-hydroxysteroid dehydrogenase activity in animal tissues

(76) Inventors: Gloria Chan Koo, Woodbridge, NJ (US); Kashmira Shah, Fords, NJ (US); Jianying Xiao, Monmouth Junction, NJ (US)

Correspondence Address: MERCK AND CO., INC, P O BOX 2000, RAHWAY, NJ, 07065-0907, US

(21) Appl. No.: 10/545,837

(22) PCT Filed: Feb. 17, 2004
(86) PCT No.: PCT/US04/04734

§ 371 Date: Aug. 17, 2005

Related U.S. Application Data
(60) Provisional application No. 60/449,381, filed on Feb. 21, 2003.

(51) Int. Cl. A61K 49/00; G01N 33/92

(52) U.S. Cl. 424/9.2; 436/71

(57) ABSTRACT

A novel method is provided to measure 11β-hydroxysteroid dehydrogenase activity in intact whole animal tissues in the presence of systemically or ex vivo administered inhibitors of the enzyme. Inhibitors of the type 1 isoform (11β-HSD1) may be useful to treat type 2 diabetes, Metabolic Syndrome, and other metabolic disorders.

### FIELD OF THE INVENTION

The present invention is concerned with a novel method to measure 11β-hydroxysteroid dehydrogenase activity in intact whole animal tissues in the presence of systemically administered inhibitors of the enzyme. The method also provides a pharmacodynamic assessment of inhibitor exposure in vivo.

### BACKGROUND OF THE INVENTION

Excessive levels of glucocorticoids can cause metabolic complications. In the Metabolic Syndrome, obesity is thought to promote insulin resistance, diabetes, dyslipidemia, hypertension, and increased cardiovascular risk. The best predictor of Metabolic Syndrome is not overall fat mass but rather visceral adiposity. Prolonged systemic exposure to glucocorticoids induces fat redistribution toward the viscera and pathological sequelae closely resembling the Metabolic Syndrome.

Though the Metabolic Syndrome is not generally associated with increased systemic concentrations of glucocorticoids, hormone actions on target tissues depend on intracellular metabolism as well as on circulating glucocorticoid levels. In particular, the enzyme 11β-hydroxysteroid dehydrogenase type 1 (11β-HSD1) plays a central role in regulating intracellular concentrations of glucocorticoids by regenerating the active molecules (cortisol and corticosterone in humans and rodents, respectively) from the inactive 11-keto precursors (cortisone and 11-dehydrocorticosterone) [see J. R. Seckl, et al., “11β-HSD type 1 —a tissue-specific amplifier of glucocorticoid action, Endocrinology, 142: 1371-1376 (2001) and Y. Kotelevtsev, et al., “11β-Hydroxysteroid dehydrogenases: key modulators of glucocorticoid action in vivo.” Curr. Opin. Endocrinol. Diabetes, 6: 191-198 (1999)] A second isoform of the enzyme 11β-hydroxysteroid dehydrogenase type 2 (11β-HSD2), which has 20% homology to 11β-HSD1, inactivates cortisol by converting it into cortisone. Over the past several years a substantial body of data has been accumulated from both murine genetic models and human studies implicating 11β-HSD1 as a driver of Metabolic Syndrome.

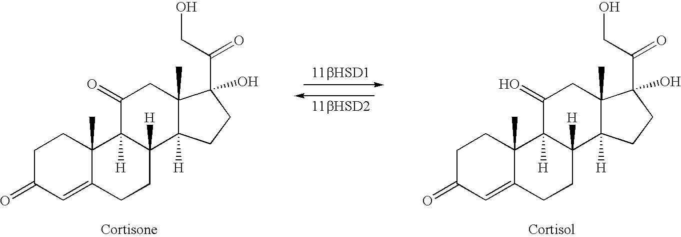

11β-HSD1-knockout mice develop normally, and are viable, fertile, and normotensive. Moreover, the model demonstrates that 11β-HSD1 is the major 11β-reductase since adrenalectomized 11β-HSD1-knockouts cannot convert 11-dehydrocorticosterone to active corticosterone. The knockout mice have impaired induction of key gluconeogenic enzymes, decreased hyperglycemic response to stress or obesity, increased insulin sensitivity and improved glucose tolerance. These findings are consistent with the increased insulin sensitivity observed in normal human volunteers treated with the HSD inhibitor carbenoxolone. The null mice also show a higher increase in diet-dependent in HDL levels, compared to wild type controls. When backcrossed to the obesity sensitive C57BL6 background, deletion of 11β-HSD1 also inhibits diet-induced weight gain. It is important to note that the knockout perturbs the BPA axis, increasing plasma levels of corticosterone and ACTH by two- to three-fold, respectively, during nadir in the diurnal cycle while still having favorable effects on Metabolic Syndrome. These results demonstrate that it is the intracellular, not the systemic, levels of glucocorticoids which, regulate the metabolic parameters.

Transgenic mice which overexpress rat 11β-HSD1 selectively in adipose tissue show all of the sequelae of Metabolic Syndrome. These mice develop visceral adiposity which is exacerbated by high-fat diet. They have pronounced insulin-resistant diabetes, exhibit hyperlipidemia, and are hypertensive. The animals have increased adiposity, and increased levels of corticosterone in adipose. However, they do not have improved circulating levels of corticosterone. Most importantly, the phenotype is produced by levels of 11β-HSD1 overexpression equivalent to, or in fact slightly less than, the increases in 11β-HSD1 activity observed in the adipose from obese humans. There is a 2.7-fold increase in 11β-HSD1 activity in the adipose of these mice, and 3-fold and 3.5-fold increases in 11β-HSD1 mRNA and activity in adipose of obese versus lean humans. Transgenic overexpression of 11P-HSD1 in the liver also induces symptoms of Metabolic Syndrome, although the extent of the phenotype appears to be somewhat milder than that produced by overexpression in adipose. Taken together, the human and murine data suggest that inhibition of 11β-HSD1 may be a useful therapeutic target for Metabolic Syndrome.

The type 1 isoform is also highly expressed in the liver. Gluconeogenes is in the liver is reduced when the 11β-HSD1 gene is knocked-out resulting in lower fasting glucose levels [Y. Kotelevtsev et al., “11β-HSD type 1 knockout mice show attenuated glucocorticoid-inducible responses and resist hyperglycemia on obesity or stress,” Proc. Natl. Acad. Sci., 94: 14924-14929 (1997)].

Additional evidence also supports the hypothesis that inhibition of 11β-HSD1 may constitute a useful approach to the treatment of type 2 diabetes without the risk of hypertension [for example, see T. Barf, et al., “Arylsulfonamidothiazoles as a New Class of Potential Antidiabetic Drugs. Discovery of Potent and Selective Inhibitors of the 11β-Hydroxysteroid Dehydrogenase Type 1,” J. Med. Chem., 45: 3813-3815 (2002)].

Only a limited number of potentially therapeutically useful inhibitors of 11β-HSD1 has been reported. For example, a class of arylsulfonamidothiazoles has been disclosed in International Patent Publications WO 01/90092 (Nov. 29, 2001) and WO 01/90094 (Nov. 29, 2001), assigned to Biovitrum AB, and published in J. Med. Chem., 45: 3813-3815 (2002). In these publications, the ability of the disclosed compounds to inhibit human or mouse 11β-HSD1 activity was assessed using an in vitro scintillation proximity assay (SPA) with recombinant human or murine enzyme with a substrate/cofactor mixture of tritiated cortisone/NADPH and different concentrations of inhibitor.

Cells isolated from adipose tissue or tissue homogenates have also been employed to quantify 11β-HSD1 activity when exposed in vitro to inhibitors [see U.S. Pat. No. 6,368,816 (Apr. 9, 2002) assigned to The University of Edinburgh]. However, this in vitro method suffers from the disadvantage that cells have to be isolated in order to measure enzyme activity and therefore does not accurately assess inhibitor exposure in vivo.

The present invention provides a novel method to measure enzyme activity of 11β-hydroxysteroid dehydrogenase in intact primary animal tissues without the need to supplement cofactors for the enzyme. The present assay provides for a pharmacodynamic assessment of inhibitor exposure in vivo with little disturbance of the equilibrium achieved in situ.

### SUMMARY OF THE INVENTION

The present invention is concerned with a novel pharmacodynamic assay useful to measure the ability of a systemically administered compound to modulate the interconversion between 11-keto and 11β-hydroxy steroid hormones mediated by 11β-hydroxysteroid dehydrogenase in various tissues of a whole animal. Inhibitors of the type 1 isoform (11β-HSD1) may be useful to treat type 2 diabetes, Metabolic Syndrome, and other metabolic disorders.

### BRIEF DESCRIPTION OF THE FIGURES

FIG. 1 shows the inhibition by Compound A of the conversion of [³H]-cortisone to [³H]-cortisol in three different mouse tissues, 4 hours after oral dosing of the compound at 1, 3, and 10 milligrams per kilogram (mpk). CPM represents counts-per-minute of [³H]-cortisol obtained in the scintillation proximity assay (SPA).

FIG. 2 shows the inhibition by Compound B of the conversion of [³H]-cortisone to [³H]-coltisol in three different rat tissues, 19 hours after oral dosing of the compound at 60 mpk. CPM represents counts-per-minute of [³H]-cortisol obtained in the scintillation proximity assay (SPA).

FIG. 3 shows the in vitro inhibition by Compound C of the conversion of [³H]-cortisone to [³H]-cortisol in two different rhesus monkey tissues. 1 μM of Compound C was added to the tissues 15 min prior to the addition of [³H]-cortisone. CPM represents counts-per-minute of [³H]-cortisol obtained in the scintillation proximity assay (SPA).

### DETAILED DESCRIPTION OF THE INVENTION

The present invention provides an ex vivo assay to measure a compound's ability to modulate 11β-hydroxysteroid dehydrogenase enzyme activity as assessed by the conversion of a steroid hormone substrate for the enzyme to its corresponding steroid hormone product. The assay of the present invention comprises the steps of:
(a) dosing said compound in a whole animal;
(b) removing tissue to be assayed from said whole animal;
(c) adding culture medium containing a steroid hormone substrate for said hydroxysteroid dehydrogenase to said tissue;
(d) incubating said culture medium containing said steroid hormone substrate;
(e) harvesting said culture medium; and
(f) assessing the extent of conversion of said added steroid hormone substrate to steroid hormone product.

In one embodiment of the assay of the present invention, the 11β-hydroxysteroid dehydrogenase is 11β-hydroxysteroid dehydrogenase type 1. In a second embodiment of the assay of the present invention, the 11β-hydroxysteroid dehydrogenase is 11β-hydroxysteroid dehydrogenase type 2.

Substrates for the 11β-HSD type 1 isoform include the 11-keto steroid hormones cortisone, dehydrocorticosterone, and prednisone, which are converted by the enzyme into cortisol, corticosterone, and prednisolone, respectively.

Substrates for the 11β-HSD type 2 isoform include the 11β-hydroxy steroid hormones cortisol, corticosterone, and prednisolone, which are converted by the enzyme into cortisone, dehydrocorticosterone, and prednisone, respectively.

The test compound to be evaluated is systemically administered to the whole animal. Systemic administration may be either by the oral or parenteral route. Parenteral administration may be by intravenous (IV), subcutaneous (SC), or intraperitoneal (IP) route. The length of exposure of the test compound in the whole animal is from about 10 minutes to about 3 days after dosage. Compounds can also be repeatedly dosed to the animals for weeks to months duration. In one embodiment the length of exposure is from about one hour to about 24 hours.

In a third embodiment of the present invention the whole animal is selected from the group consisting of rat, mouse, rabbit, guinea pig, dog, non-human primate, and human. In a class of this embodiment, the whole animal is a rat, mouse, or non-human primate. In another class of this embodiment, the non-human primate is a rhesus monkey.

In a fourth embodiment, the tissue to be assayed from the whole animal is selected from the group consisting of liver, brain, muscle, lung, pancreas, kidney, blood, and adipose. The tissue is removed from the whole animal by surgical procedure, dissection, or biopsy.

In a fifth embodiment of the present invention, the tissue removed from the whole animal is weighed prior to addition of a certain volume of the culture medium. In a class of this embodiment the ratio of the weight of the tissue (in milligrams) to volume of culture medium added (in milliliters) is from about 1:3 to about 1:10. In a class of this embodiment, the ratio is about 1:5. The tissue may then be minced as with scissors prior to incubation. Incubation is effectively carried out at 37° C. under a carbon dioxide atmosphere for about 10 minutes to about 24 hours depending on the tissue. Cells are not isolated from the tissues, nor is any homogenization carried out.

Harvesting comprises decanting off the supernatant followed by optional centrifuging of the supernatant and decanting to remove cellular debris.

The extent of conversion of enzyme substrate to enzyme product in the supernatant is then measured either by high-performance liquid chromatography (HPLC) or by using an antibody to enzyme product using a scintillation proximity assay (SPA). For example, the BPLC method to assay 11β-HSD1 activity uses either cold cortisone or [³H]-cortisone. The SPA assay utilizes [³H]-cortisone and is described in the Supporting Information for the J. Med. Chem., 45: 3813-3815 (2002) article available via the Internet at http://pubs.acs.org. The contents of this article are incorporated by reference herein in their entirety.

The following Examples are provided for purposes of illustration only and are not intended to limit the method of the present invention to the specific conditions for conducting the assay.

### EXAMPLE 1

The Example detailed below detects the enzymatic activity of 11β-HSD1, the conversion of cortisone to cortisol, in the absence or presence of inhibitor compounds. If an inhibitor compound for 11β-HSD1 activity is present, conversion will be inhibited, and the degree of inhibition is a measure of the effect of the inhibitor at a respective concentration.

General Protocol:

1. Animals were dosed once or multiple times with vehicle or test compounds administered by the oral (PO), IV, SC or IP route.

2. After a period of time, (10 min to 60 days), the animals were euthanized and then exsanguinated by cardiac bleeding. Tissues of any type, such as for example, adipose, liver, brain, muscle, etc., were removed, and put in 24 well plates. They were kept on ice and weighed (about 200 mg).

3. RPMI, supplemented with 5% fetal calf serum (Sigma) and 1% penicillin-streptomycin (GIBCO), and containing 15-20 nM [³H]-cortisone was added to each piece of tissue. The total volume (in mL's) added was equivalent to about 5 times the mass of tissue (in milligrams).

4. Tissue was then minced into 2-3 mm pieces with scissors, and subsequently incubated at 37° C. in a 5-7% CO₂ atmosphere for 10 min to 3 h, depending on the tissue. Cells were not isolated from the tissues, nor was any homogenization performed.

5. At the end of the incubation period, supernatant was collected. In some cases the supernatant was spun at 10,000 rpm (Eppendorf centrifuge) for about 2 min to remove cellular debris.

6. The amount of conversion of [³H]-cortisone to [³H]-cortisol in the supernatant was then measured either by HPLC methods or by scintillation proximity assay (SPA) using a commercially available antibody to cortisol.

Materials and Methods:

Animals:

Various strains of rat, mouse, rabbit, guinea pig, dog, and non-human primates were used. Human tissues can also be used to test for compound inhibition which can be obtained, for example, by punch biopsy. Animals (e.g. mice or rats) were dosed with vehicle or 0.1 to 1000 mg/kg of test inhibitor compound. A more preferred dosing range is 1 to 100 mg/kg of test inhibitor compound. The test inhibitor compound was administered by the oral, IV, SC or IP route. After a period of time, (10 min to 3 days), the animals were euthanized and then exsanguinated by cardiac bleeding. Tissues were removed and placed in 24 well-plates. They were kept on ice, and weighed (about 200 mg). RPMI (GIBCO), supplemented with 5% fetal calf serum (Sigma) and 1% penicillin-streptomycin (stock solutions from GIBCO), and containing 15-20 nM [³H]-cortisone was added to each piece of tissue. The total volume (mL's) added was equivalent to about 5 times the mass of tissue (milligrams). Tissue was then minced into 2-3 mm pieces with scissors, and subsequently incubated at 37° C. in a 5-7% CO₂ atmosphere for 10 min to 3 h, depending on the tissue. Cells were not isolated from the tissues, nor was any homogenization performed. At the end of the incubation period, supernatant was collected. In some cases, supernatant was spun at 10,000 rpm (Eppendorf centrifuge) for about 2 min to remove cellular debris. The amount of conversion of [3H]-cortisone to [³H]-cortisol in the supernatant was then measured either by HPLC methods or by using an antibody which specifically binds to [3H]-cortisol. In the latter assay, the [3H]-cortisol was captured by monoclonal antibody to cortisol (Biostride, Inc.) and protein A-coated scintillation impregnated proximity beads [Amersham/Pharmacia, RPN-143]. The amount of radioactive product, [³H]-cortisol, captured on the beads was determined in a microplate liquid scintillation beta counter.

The following conditions were used for the HPLC assay of the conversion of cortisone to cortisol:

200 μL media supernatants were extracted with 1 mL of ethyl acetate. Samples were air dried and resuspended in DMSO containing 16 μg/mL (1:1) of unlabeled cortisone to cortisol (Sigma, St. Louis, Mo.). Samples were injected into a Shimadzu SCL-10A VP HPLC system using an Inertsil 5-beta M ODS2 column. 100 μL of sample was injected onto the Inertsil column and eluted using 70% Buffer A/30% Buffer B to 40% buffer A/60% buffer B (Buffer A: 10% Methanol+90% Water+0.05% trifluoroacetic acid; Buffer B: 90% Methanol+10% Water+0.05% trifluoroacetic acid). [³H]-steroids were detected by a beta-RAM radiochromatography detector (IN/US Systems, Tampa, Fla.). Data was analyzed using WIFLOW (IN/US Systems) and subsequently exported to Microsoft Excel. Relative levels of conversion of [³H]-cortisone to [3H]-cortisol were used to determine activity and accompanying inhibition by compounds.

Results:

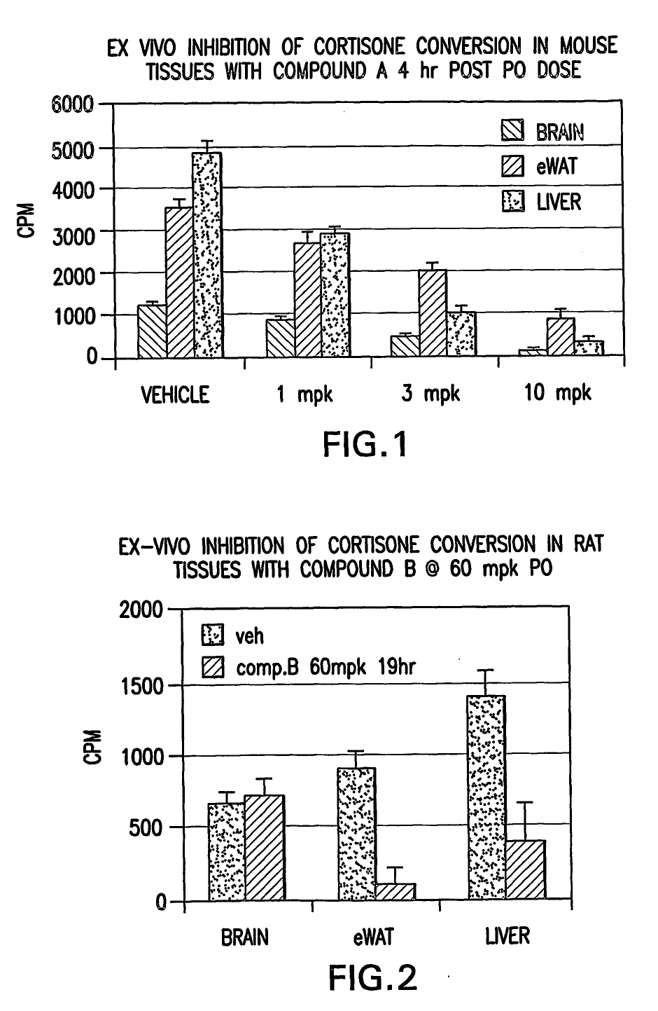

Compound A was given by oral gavage, and 4 h later, mice (n=4) were euthanized and tissues were removed. Liver, brain, and epididymal white adipose tissue (eWAT) were processed as described above. Liver tissues were incubated for 10 min at 37° C., and brain and adipose tissues were incubated for 2 h, and supernatants were collected and assayed for cortisol conversion. FIG. 1 shows the amount of counts obtained and the extent of inhibition of the conversion of cortisone to cortisol by three different doses of Compound A.

### EXAMLE 2

FIG. 2 shows the activity of Compound B in three different rat tissues when dosed orally by gavage at 60 mpk for 19 hours before euthanasia. CPM represents counts-per-minute of [³H]-cortisol in the scintillation proximity assay (SPA).

### EXAMPLE 3

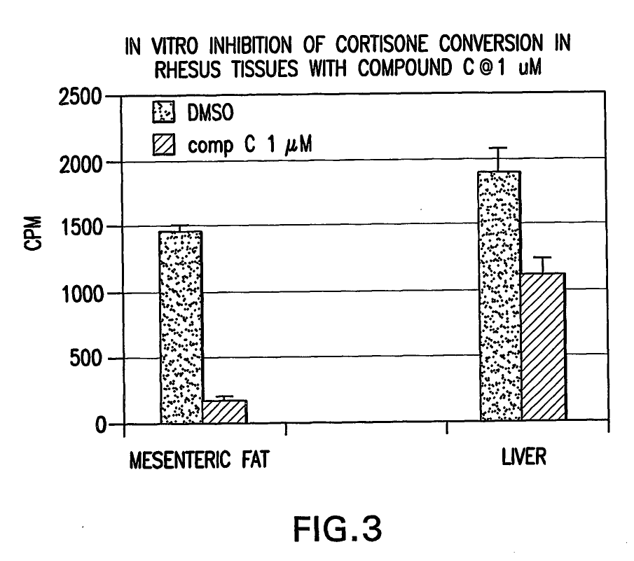

FIG. 3 shows the activity of Compound C in two different rhesus monkey tissues when incubated for 15 min at 37° C. before addition of the [³H]-cortisone. CPM represents counts-per-minute of [3H]-cortisol in the scintillation proximity assay (SPA).

It will be apparent to those skilled in the art that various modifications and variations can be made to the protocol without departing from the scope of the invention. Thus, the present invention includes modifications and variations that are within the scope of the claims and their equivalents.

What is claimed is:

1. A process for assaying the ability of a compound to inhibit the activity of 11β-hydroxysteroid dehydrogenase in whole animal tissue comprising the steps of:
(a) dosing said compound in a whole animal;
(b) removing tissue to be assayed from said whole animal;
(c) adding culture medium containing a steroid hormone substrate for said hydroxysteroid dehydrogenase to said tissue;
(d) incubating said culture medium containing said steroid hormone substrate;
(e) harvesting said culture medium; and
(f) assessing the extent of conversion of said added steroid hormone substrate to steroid hormone product.

2. The process of claim 1 wherein said 11β-hydroxysteroid dehydrogenase is 11β-hydroxysteroid dehydrogenase type 1.

3. The process of claim 1 wherein said 11β-hydroxysteroid dehydrogenase is 11β-hydroxysteroid dehydrogenase type 2.

4. The process of claim 2 wherein said steroid hormone substrate is cortisone, dehydrocorticosterone, or prednisone.

5. The process of claim 3 wherein said steroid hormone substrate is cortisol, corticosterone, or prednisolone.

6. The process of claim 1 wherein said compound is systemically administered to said whole animal.

7. The process of claim 6 wherein said systemic administration is by the oral or parenteral route.

8. The process of claim 1 wherein said whole animal is selected from the group consisting of rat, mouse, rabbit, guinea pig, dog, non-human primate, and human.

9. The process of claim 8 wherein said whole animal is a rat, mouse, or non-human primate.

10. The process of claim 1 wherein said whole animal tissue is selected from the group liver, brain, muscle, lung, pancreas, kidney, blood, and adipose.

11. The process of claim 1 wherein said conversion of steroid hormone substrate to steroid hormone product is assessed by a high-performance liquid chromatography method or by a scintillation proximity assay method.
